(19) United States
(12) Patent Application Publication
(10) Pub. No.: US 2022/0380837 A1
(43) Pub. Date: Dec. 1, 2022

(54) METHOD FOR PREPARING PROBE TARGETING TARGET NUCLEIC ACID TARGET

(71) Applicant: Tsinghua University, Beijing (CN)

(72) Inventors: Xu ZHANG, Beijing (CN); Jing NIU, Beijing (CN); Juntao GAO, Beijing (CN); Wei LUO, Foshan (CN); Yong JI, Foshan (CN)

(21) Appl. No.: 17/439,023

(22) PCT Filed: Mar. 13, 2020
(86) PCT No.: PCT/CN2020/079121

§ 371 Date: May 3, 2022

(30) Foreign Application Priority Data
Mar. 15, 2019 (CN) 201910197017.3

(51) Int. Cl. C12Q 1/6834; C12Q 1/6841

(52) U.S. Cl. CPC C12Q 1/6834; C12Q 1/6841

(57) ABSTRACT

The present invention relates to a method for preparing probes for target nucleic acids targets. This method includes: a) obtaining a target DNA sequence of interest; b) adding adapter sequences to both ends of a fragmented DNA sequence while fragmenting the target DNA sequence by using transposase; and c) obtaining the fragmented DNA sequence by using the adapter sequences to generate the probes. The method provided by the present invention can efficiently, easily, and accurately mark the position of the genome at the level of one kilobase resolution.

### RELATED APPLICATIONS

The present application is a U.S. National Phase of International Application Number PCT/CN2020/079121 filed Mar. 13, 2020, and claims priority to Chinese Application Number 201910197017.3 filed Mar. 15, 2019.

### INCORPORATION BY REFERENCE

The sequence listing provided in the file entitled GNPHZ20013_SQL_rev1.txt, which is an ASCII text file that was created on Apr. 28, 2022, and which comprises 9,391 bytes, is hereby incorporated by reference in its entirety

### TECHNICAL FIELD

The present invention relates to the field of molecular biology, particularly to a method of preparing probes for targets on nucleic acids of interest.

### BACKGROUND

Fluorescent in situ Hybridization (FISH) that can provide spatial position information of labeled sites in a cell nucleus by means of the sequence and fluorescence of a hybridization probe, is complementary to various biological techniques (e.g. 4C, 5C, Hi-C, ChIA-PET, etc.) based on Chromatin Conformation Capture (3C) all the time, and becomes one of the indispensably important techniques for studying chromatin structures. The traditional FISH technique generally uses a complete genomic fragment (usually BAC, PAC, YAC, etc.) derived from the target species as a template, fragments it through the action of bio-enzyme, and then performs fluorescence labeling to make hybridization probes. In fixed cells, specific genomic fragments are fluorescently labeled and imaged through the principle of base complementary pairing to obtain specific nucleic spatial information. However, the traditional FISH techniques, limited by the characteristics of BAC and other templates, have the disadvantages of long preparation time, a great number of templates needed, low gene resolution (100-200 Kb), repeated fragments contained in clones, adding species-specific Cot-1 DNA required and the like. In the research of chromatin structure, the traditional FISH technique shows a low applicability of the label for a large number of interactions less than 200 Kb and the research on species without commercialized Cot-1 DNA is even more difficult. Therefore, there is an urgent need to develop a FISH method which is fast and efficient, requires a low number of templates, has a high genomic resolution and does not require Cot-1 DNA to replace the current traditional FISH technique solution.

So far, among novel FISH techniques that have been reported, the Oligopaint technique, HD-FISH technique, CasFISH technique and MD-FISH technique have all been optimized to different extents for the abovementioned four points, with major advancements lie in improved genomic resolution (2.5-10 Kb) and without the need to add Cot-1 DNA to suppress repeated sequences. However, some of the four techniques are costly, complex in preparation and not cost-effective, and some need bioinformatic tools to dig out a proper probe sequence, which are difficult for ordinary laboratories to apply these techniques directly.

### SUMMARY

The present invention provides a method of preparing probes for targets on the nucleic acids of interest.

The method of preparing probes for targets on nucleic acids of interest provided by the present invention includes:

a) obtaining a target DNA sequence of interest;

b) using transposase to fragment the target DNA sequence and add adapter sequences to both ends of the fragmented DNA sequence; and

c) using the adapter sequences to obtain the fragmented DNA sequence to generate the probes.

An important advantage is that this method does not rely on or seldomly rely on the specificity of the initial DNA sequence, and this method can effectively remove the regions containing undesired sequences (especially repeated sequences) from the original DNA sequences of target genomic loci, so that the method does not rely on the species-specific Cot-1 DNA to block repeated fragments.

An important advantage is that the amount of DNA template required for preparing probes in this method is about 50 ng (for example, 30 ng, 35 ng, 40 ng, 45 ng, 55 ng, 60 ng), which is much lower than 1 μg for traditional FISH; while for one site, a large number of probes can be prepared after only one Tn5 high-efficiency transposase fragmentation. The process is simple, efficient, and cost-effective;

An important advantage is that the implementation of this method requires the operator to have only basic molecular biology techniques, therefore the technical threshold is relatively low;

An important advantage is that this method is suitable for analyzing chromatin interactions within 100 Kb;

An important advantage is that this method has a genomic resolution up to about 1 kb labeling capability.

The target DNA sequence in the present invention can be derived from any sample containing target DNA.

The term “sample” is used in its broadest meaning. In one meaning, it is meant to include cells (for example, human, bacteria, yeast and fungi), tissue or living organisms, or samples or cultures obtained from any source, and biological samples. The biological samples can be obtained from animals (including people) and refer to biological materials or compositions found therein, including but not limited to bone marrow, blood, blood serum, blood platelet, plasma, interstitial fluid, urine, cerebrospinal fluid, nucleic acids, DNA, tissue and their purified or filtered forms. However, these examples shall not be constrained as limiting the types of the samples that can be used in the present invention.

In some embodiments, the sample is a whole genomic DNA.

In some embodiments, the transposase is highly active.

As used herein, the term “nucleic acid” refers to any molecule comprising nucleic acids, including but not limited to DNA or RNA. The term encompasses sequences that include any known base analogs of DNA and RNA, including but not limited to: 4-acetylcytosine, 8-hydroxyl-N6-methyladenosine, aziridinyl cytosine, pseudoisocytosine, 5-(carboxyl hydroxyl methyl) uracil, 5-fluorouracil, 5-bromouracil, 5-carboxymethyl amino methyl-2-thiouracil, 5-carboxymethyl amino methyl uracil, dihydrouracil, inosine, N6-isopentenyl adenine, 1-methyl adenine, 1-methyl pseudouracil, 1-methyl guanine, 1-methyl inosine, 2,2-dimethyl guanine, 2-methyl adenine, 2-methyl guanine, 3-methyl cytosine, 5-methyl cytosine, N6-methyl adenine, 7-methyl guanine, 5-methyl amino methyl uracil, 5-methoxyl amino methyl-2-thiouracil, β-D-mannosyl Q nucleoside, 5′-methoxyl carboxyl methyl uracil, 5-methoxyl uracil, 2-methylmercapto-N6-isopentenyl adenosine, Urine-5-oxyacetic acid methyl ester, Urine-5-oxyacetic acid, oxybutoxosine, pseudouracil, Q nucleoside, 2-thiocytosine, 5-methyl-2-thiouracil, 2-thiouracil, 4-thiouracil, 5-methyl uracil, N-uracil-5-oxy methyl acetate, uracil-5-oxyl acetic acid, pseudouracil, Q nucleoside, 2-thiocytosine and 2,6-diamino purine.

The target nucleic acid detected by the probes prepared by the present invention is generally a DNA sequence, but various RNA sequences or DNA-RNA mixed sequences are not excluded, for example: mRNA sequences obtained by transcribing from the target DNA sequence of interest.

In some embodiments, the target DNA sequence of interest is obtained by removing the region containing the undesired sequences from the initial sequence.

As used herein, the term “initial sequence” refers to a fragment of a genomic sequence of the target biological tissue. The term “region containing the undesired sequence” refers to a region not containing (for example, 85%, 90%, 91%, 92%, 93%, 94%, 95%, 96%, 97%, 99% or 100% of) undesired nucleic acids substantially. The undesired nucleic acids include but are not limited to repeated nucleic acids, nonconservative sequences, conserved sequences, GC-rich sequences, AT-rich sequences, secondary structures, noncoding sequences (for example, promoters, enhancers, etc.) or coded sequences.

In some embodiments, the undesired region is selected from the repeated sequences.

In some embodiments, the excluding method is to amplify the target DNA sequence of interest.

In some embodiments, the amplification is PCR amplification.

In some embodiments, the region containing the undesired sequences is at least 100 bp, or 120 bp, 130 bp, 140 bp, 150 bp, 160 bp, 170 bp, 180 bp, 190 bp, 200 bp, 250 bp, 300 bp, 350 bp, 400 bp, 450 bp, 500 bp, 600 bp, 700 bp, 800 bp, 900 bp, 1,000 bp, 1,500 bp, 2,000 bp, 3,000 bp, 4,000 bp, 5,000 bp, 6,000 bp, 7,000 bp, 8,000 bp, 9,000 bp, 10,000 bp, 20,000 bp, 30,000 bp, 40,000 bp or 50,000 bp.

In some embodiments, the transposase is selected from one of or any combination of Tn1, Tn2, Tn3, Tn4, Tn5, Tn6, Tn7, Tn9, Tn10, Tn551, Tn971, Tn916, Tn1545, Tn1681, Tgf2, Tol2, Himar1 and HARBI1.

Tgf2 and Tol2 are from hAT family, Himar1 is from Tcl/Mariner family and HARBI1 is from PIF/Harbinger family.

In some embodiments, the probes are labeled.

The term “label” used herein refers to any atom or molecule that can be used to provide a detectable (preferably quantifiable) effect and can be attached to a nucleic acid or protein. The labels include but are not limited to dyes; radioactive labels such as ³²P; binding moieties such as biotin; haptens such as digoxin; luminescent, phosphorescent or fluorescent moieties; and independent fluorescent dyes or fluorescent dyes combined with parts of the emission spectrum that can be suppressed or shifted by fluorescence resonance energy transfer (FRET). The label can provide signals that can be detected through fluorescence, radioactivity, colorimetry, gravimetry, X-ray diffraction or absorption, magnetism, enzymatic activity and the like. The label may be a charged moiety (positive or negative) or alternatively, it may be neutral. The label can include nucleic acids or protein sequences or a combination thereof as long as the sequences containing the label are detectable. In some embodiments, the nucleic acids are directly detected (for example, the sequence is read directly) without being labeled.

In some embodiments, the label is a fluorophore, a colorimetric label, a quantum dot, a biotin and other label molecules for detection (for example, alkyne groups for Raman diffraction imaging, cycloolefins for click reaction, priming groups for polymer labeling), and can further be selected from a polypeptide/protein molecule, LNA/PNA, unnatural amino acids and their analogs thereof (for example peptoid), unnatural nucleic acids and an analogs thereof (for example nucleotides) and a nanostructure (including inorganic nanoparticles, NV-center, aggregation/assembly induced emission molecules, rare earth ion ligand molecules, and polymetallic oxygen clusters, etc.). The colorimetric label refers to a label that can be used in colorimetric analysis.

In some embodiments, the label is a fluorophore.

In some embodiments, the fluorophore can be selected from fluorescein dyes, rhodamine dyes, and cyanine dyes.

In some embodiments, the fluorescein dyes include standard fluorescein and its derivative, for example, fluorescein isothiocyanate (FITC), hydroxyl fluorescein (FAM), and tetrachlorofluorescein (TET), etc.

In some embodiments, the rhodamine dyes include R101, tetraethyl rhodamine (RB200), carboxyl tetramethyl rhodamine (TAMRA), etc.

In some embodiments, the cyanine dyes are mainly selected from two types: one is thiazole orange (TO), oxazole orange (YO) series and their dimmer dyes and the other is polymethenyl series cyanine dyes.

In some embodiments, the fluorophore can further be selected from the following dyes: diphenylethene, naphthalimide, coumarins, acridine, pyrene, etc.

The fluorophore is generally labeled at the 5′ terminal of a primer or probe sequence, but it can also be placed at the 3′ terminal by changing a modifier bond (for example, —OH or —NH bond).

In some embodiments, in Step c), the method of generating probes includes amplification, cloning, synthesizing and a combination thereof.

The term “amplifying or amplification” in the context where the term “nucleic acids” is used refers to generating a plurality of copied polynucleotides or a part of polynucleotides, generally starting from the small amount of polynucleotides (for example, as few as a single polynucleotide molecule), wherein the amplification product or amplicon usually detectable. Amplification of polynucleotide includes various chemical and enzymatic methods. In polymerase chain reaction (PCR), rolling circle amplification (RCA) or ligase chain reaction (LCR) processes, the amplification means to generate a plurality of DNA copies from one or more copies of the target DNA or template DNA molecules. Amplification is not limited to the strict duplication of the initiating molecule. For example, it is a form of amplification to generate a plurality of cDNA molecules from a limited amount of RNAs in a sample by using reverse transcription RT-PCR. In addition, in the transcription process, it is also a form of amplification to generate a plurality of RNA molecules from a single DNA molecule.

In some embodiments, in Step c), the method of generating probes is to amplify the fragmented DNA sequence by using the primers capable of binding to the adaptor sequence.

The term “primer” refers to an oligonucleotide. Regardless of whether it occurs naturally in a purified restrictive digest or is generated by synthesis, the oligonucleotide should be served as an initial point of synthesis to play a role under the condition of inducing synthesis of an extended product of the primer complementary to a nucleic acids chain (for example, in the presence of nucleotide and inducer such as DNA polymerase and at the proper temperature and pH value). The primer is preferably single-stranded for the maximum efficiency of amplification and it can optionally be double-stranded. If the primer is double-stranded, the primer is processed to separate the strands thereof first before being used for preparing the extended products. Preferably, the primer is an oligodeoxyribonucleotide. The primer shall be long enough to initialize the synthesis of the extended products in the presence of an inducer. The exact length of the primer will depend on many factors, including temperature, primer source and method used. For example, in some embodiments, the rang of primers is 10-100 or more nucleotides (e.g., 10-300, 15-250, 15-200, 15-150, 15-100, 15-90, 20-80, 20-70, 20-60, 20-50 nucleotides, etc).

In some embodiments, the primer contains other sequences that do not hybridize to the target nucleic acids. The term “primer” includes chemically modified primers, fluorescently modified primers, functional primers (fusion primers), sequence specific primers, random primers, primers with specificity and random sequences, and DNA and RNA primers.

In some embodiments, the primer is labeled.

In some embodiments, the label is defined by the abovementioned term “label”.

In some embodiments, the label is selected from fluorophores, colorimetric labels, quantum dots or biotin; preferably fluorophores.

According to yet another aspect of the present invention, the present invention also relates to methods for hybridization assay, which include generating probes by using the method described above and contacting the target nucleic acids with the probes.

The term “hybridization” used herein refers to the pairing of complementary nucleic acids. Hybridization and hybridization strength (i.e., the bonding strength between the nucleic acids) are affected by such factors as the complementary degree between nucleic acids, the stringency of conditions involved, the Tm of the hybrid formed, and the G:C ratio in the nucleic acids, etc. The paired single molecule containing the complementary nucleic acids in its structure thereof is “self-hybridized”.

In some embodiments, the hybridization assay is in situ hybridization.

Preferably, one, two or more kinds of probes can be used for the assay method. The two or more kinds of probes refer to two or more kinds of probes for the target DNA sequence. The DNA probes for each of the target sequences can be composed of one, two or more DNA sequence fragments located in the target sequence DNA.

When there are two or more kinds of probes, each kind of probes will display different colors after being labeled. At least one kind of probes is a Tn5-FISH probe, and other applicable probes can be either Tn5-FISH probes with different colors or probes with different colors prepared by other methods, including but not limited to BAC cloning probes, YAC cloning probes, probes prepared from Cosmid and Fosmid, artificially synthesized probes, Oligopaint probes, and RNA-FISH probes, etc.

Preferably, the in situ hybridization refers to 3D FISH labeling the in fixed target cells.

According to yet another aspect of the present invention, the present invention also relates to a method for hybridization assay, which includes generating probes by using the method described above and contacting the target nucleic acids with the probes.

### BRIEF DESCRIPTION OF THE DRAWINGS

FIG. 1 illustrates a flow diagram of an embodiment of the present invention.

FIGS. 2a-2d illustrates, in one embodiment of the present invention, a comparison of combination of Tn5-FISH and traditional BAC FISH in WT mESC cells and Platr22-KO mESC cells to verify the labeling specificity of the Tn5-FISH genomic site.

The BAC probes (green) and the Tn5-Platr22 probes (red, FIG. 2a, FIG. 2b) or the Tn5-GM19705 probes (red, FIG. 2c, FIG. 2d) are hybridized simultaneously in the WT mESC cells (FIG. 2a, FIG. 2c) or Platr22-KO mESC cells (FIG. 2b, FIG. 2d).

FIG. 3 illustrates, in one embodiment of the present invention, a comparison of combination of Tn5-FISH and BAC FISH in the K562 cells to verify the resolution of 1 KB genomic loci.

FIGS. 4a-4c illustrates, in one embodiment of the present invention, multicolor Tn5-FISH verifies the predicted interactions with interaction sites at both ends of chr2: 227672028-227743852 in GM12878 cells.

FIGS. 5a-5d illustrates, in one embodiment of the present invention, a comparison of a combination of Tn5-FISH and BAC FISH in the K562 cells to verify genetic sites. In FIGS. 5a-5d, from left to right, FIG. 5a shows labeling the genetic site (red) by the Tn5 probes in Step 2, panel FIG. 5b shows labeling the genetic site (green) by the BAC probes in Step 2, panel FIG. 5c shows labeling cell nuclei by DAPI and FIG, and panel FIG. 5d shows the merged graph of the images in three channels.

### DETAILED DESCRIPTION OF THE EMBODIMENTS

The embodiments below are intended for a better understanding of the present invention, but not limiting the present invention. Unless otherwise specified, the experimental methods in the embodiments below are traditional methods. Unless otherwise specified, test materials used in the embodiments below are purchased from biochemical venders, respectively. Three repeated experiments are set for the quantitative tests in the embodiments below, and the result is a mean value.

I. Materials and Reagents

Instruments: PCR instrument (Biored), hybridization instrument and water bath kettle

Reagents: RPMI1640 culture medium (purchased from GIBCO), DMEM culture medium (purchased from GIBCO), streptomycin/penicillin antibodies (purchased from GIBCO), trypsin (purchased from GIBCO) and FBS (purchased from GIBCO). Genomic DNA extraction kit (purchased from Life Technology), Qubit DNA high sensitivity kit (purchased from Life Technology), AntiFade mounting agent (containing DAPI, purchased from Life Technology), Fixogum (purchased from Marubu), Tn5 transposase kit (purchased from Vazyme), HS-Taq (purchased from Takara), PCR product purification kit (purchased from Zymo), 37% hydrochloric acid (purchased from SINOPHARM), Tris-HCl (purchased from sigma), Triton-X 100 (purchased from sigma), ethanol (purchased from sigma), dextran sulfate (purchased from sigma), hepatic glycogen (purchased from Life Technology), 20×SSC (purchased from Life Technology), salmon sperm DNA (purchased from Life Technology), deionized formamide (purchased from Solarbio), PBS (purchased from Solarbio), 3M sodium acetate purchased from Solarbio), 4% paraformaldehyde (purchased from Solarbio), NP-40 (purchased from Solarbio) and DNase-free RNase A (purchased from Solarbio). All the primers are synthesized and provided by Ruibiotech. All the BAC clones in the present invention are purchased from Life Technology.

Cell strains: K562 cells (purchased from ATCC), GM12878 cells (purchased from ATCC) and mouse ESC cells (purchased from ATCC).

Consumables: SuperFrost glass slide (purchased from ThermoFisher), ThermoFisher 1.5# cover glass (purchased from ThermoFisher).

II. Steps of Method for Preparing the Probes

1. The amplification primer: the corresponding primers were designed for the genomic site to be labeled, and prepared via a primer synthesizing company. A fluorescently-labeled primer was synthesized according to the sequence provided by the Tn5 kit, and all the fluorescent molecules were labeled at 3′ terminal.

2. Extracting genomic DNAs: 1×10⁶ cells were taken, for each kind of cells, and DNAs were extracted according to the experimental steps of the genomic DNA extraction kit. The extracted DNAs were quantified with Qubit and stored at −20° C.

3. Obtaining probes template DNAs: 50 ng of genomic DNA was taken, the diluted primer was added into a PCR tube, and PCR was conducted in a volume of 50 microliter. The PCR conditions were as follows: at 98° C. for 3 min (at 98° C. for 30 s, at 55° C. for 30 s, at 72° C. for 3 min)×30 cycles, at 72° C. for 5 min, and kept at 4° C. A PCR product was purified and recovered by the recovery kit, quantified with Qubit, and stored at −20° C.

4. Tn5 fragmentation: 50 ng of DNA product in Step 3 was taken, and Tn5 enzyme and a reaction buffer were added to reach a total volume of 50 μL. The DNA product was treated in a 55° C. water bath kettle for 10 min, and then DNAs were purified by using the PCR product recovery kit.

5. PCR amplification and fluorescence labeling: All the products in Step 4 were put for PCR amplification. The PCR conditions were as follows: at 75° C. for 5 min (at 98° C. for 30 s, at 55° C. for 30 s, at 72° C. for 30 s)×30 cycles, at 72° C. for 5 min, and kept at 4° C. After the DNA product was purified, 50 ng was taken as a template, and the primer with a fluorescent label was used for PCR amplification & labeling. The PCR conditions were as follows: at 98° C. for 3 min (at 98° C. for 30 s, at 55° C. for 30 s, at 72° C. for 30 s)×30 cycles, at 72° C. for 5 min, and forever at 4° C. 2 μL of hepatic glycogen and 10 μL of salmon sperm DNAs were added to carry out ethanol precipitation at 80° C. below zero for 2 hours (0.1 time of 3M sodium acetate in volume and 2.5 times of absolute ethyl alcohol in volume) after the labeled product was quantified with Qubit. The probes subject to alcohol precipitation were washed for three times with 75% ethanol, resuspended with a hybridization buffer (2×SSC, 10% dextran sulfate, 50% deionized formamide) after ethanol was vaporized thoroughly, and stored at 20° C. below zero.

6. In situ hybridization: The cells were immobilized with 4% paraformaldehyde at room temperature for 10 min, and washed with 0.1M Tris-HCl for 10 min; then a membrane was penetrated with 0.5% Triton-X 100 containing 10 μg/mL RNase A and RNAs were digested; the cells were subject to water bath treatment at 37° C. for 30 min, the cells were washed with PBS for three times and treated with a hydrochloric acid solution at room temperature for 30 min. After being washed with PBS for three times, the cells were treated in a 50% deionized formamide 2×SSC solution at room temperature for 30 min, and dried after gradient ethanol dehydration. 10 microliters of probes solution (2 ng/μL) and the cells were mixed, and the mixture was sealed in the glass slide with Fixogum, and placed in a hybridization instrument for hybridization (at 75° C. for 5 min, and at 37° C. for overnight). The cells were washed for three times at room temperature with a 0.3 NP-40 2×SSC solution the next day, 5 min every time, and then mounted with the AntiFade mounting agent; the solution was sealed at the edge of the glass slide with Fixogum, and kept in a dark place at 4° C. or photographed directly.

7. Fluorescence imaging and processing: The sealed slide was photographed with a fluorescence microscope, or a confocal microscopy. The confocal microscopy (model: LSM780) of Zeiss is used in the present invention, equipped with 405, 488, 568, 594 and 647 laser and corresponding optical filter combinations, the lens being a 63× ApoPLAN NA1.4 oil immersion lens. Immersion oil is Zeiss Immersion Oil F518, and the refractive index at 25° C. is 1.515. The picture collecting software is ZEN SP2.3, and the processing software is FIJI (ImageJ core version: 1.52h).

In a specific embodiment, the present invention discloses a method of preparing a high-resolution FISH probes, comprising the steps of (as shown in FIG. 1):

(1) Obtaining of probes template by genomic PCR: A primer was designed to obtain a specific DNA fragment for a specific labeled fragment, used as probes preparation template;

(2) Fragmentation of the probes preparation template DNA: A specific amount of probe preparation template DNAs (for example, 1 ng, 5 ng, 50 ng, 100 ng, 200 ng or 500 ng) were taken, and Tn5 highly active transposase was added for fragmentation;

(3) PCR amplification: The fragmented DNAs obtained in Step (2) were subject to PCR amplification to obtain a lot of unlabeled probes DNAs;

(4) Fluorescence labeling of the probes: The fragments obtained in Step (3) were subject to PCR amplification by the primer with the fluorescent label to add the fluorescent molecules;

(5) In situ hybridization: The fluorescent probes DNAs obtained in Step (4) and the immobilized cells of interest were subject to 3D FISH labeling; and

(6) Fluorescence imaging: The cells labeled by the FISH method were photographed and imaged microscopically.

### Embodiment 1 Preparation of Probes for the Targets on Nucleic Acids of Interest and Hybridization Assay

An example is labeling one genetic site on chromosome 12 in the K562 cells.

1. Preparation of Tn5 Probes

a) Acquisition of the DNA Fragment of the Target on Nucleic Acids of Interest

An example is labeling one genetic site on chromosome 12 in the K562 cells. A DNA sequence (as an initial sequence) containing a genetic site chr12: 52,760,000-52,790,000 was downloaded from the UCSC website (hg19). The undesired sequence was excluded from the initial sequence, wherein the undesired sequence was a plurality of repeated sequences, with a size of 200-1544 bp; a primer F: 5′ ATCCTTCCAGTGTTAGGTTGA3′ and a primer R: 5′ TTGTCAGGTCTCAACGGTCT3′ were designed in a position without the repeated sequences, wherein the length of the target DNA sequence of interest was 2580 bp. The second tube was amplified by taking PrimeSTAR HS (Premix) as an example. Wherein a PCR system is shown in Table 1, and a PCR reaction procedure is shown in Table 2. A PCR product stripe was verified by means of running 1% agarose gel, the gel was cut to recover the stripe, and the stripe was eluted with 30 μL of ddH₂O and quantified with Qubit. The stripe was stored at 20° C. below zero; and the target DNA fragments of interest were obtained. The sequence of the DNA fragment is shown in sequence 1.

TABLE 1
50 μL PCR system

| | Volume | Final | |
|---|---|---|---|
| Components | (μL) | concentration | |
| PrimeSTAR HS | 25 | 1× | |
| (Premix) | | | |
| DNA template | | ~200 | ng |
| Primer F | 2.5 | 0.5 | μM |
| Primer R | 2.5 | 0.5 | μM |

| | |
|---|---|
| ddH₂O | |
| Total volume | 50 |

TABLE 2
PCR procedure.

| Temperature | Time | |
|---|---|---|
| 98° C. | 10 | s |
| 58° C. | 15 | s |
| 72° C. | 1 Kbp/min, determined | |
| | according | |
| | to the length | |
| | of fragment | |
| Number of Cycles | 33 | |
| 72° C. | 5 | min |
| 4° C. | 10 | min |

b) Adapter Sequences were Added to Both Ends of a Fragmented DNA Sequence by Using Tn5 Transposase while Fragmenting the Target DNA Sequence

50 μL of Tn5 transposase (V50 Tn5 Enzyme 5) enzyme digestion reaction system was prepared according to Table 3, blown slowly with a pipette and mixed uniformly. The first tube was digested: a digestion reaction was conducted at a constant temperature of 55° C. for 10 min, and the tube was stored at 4° C.; the digested DNA fragments were recovered with a PCR cleanup Kit and eluted with 10 μL of ddH₂O. The fragmented DNA sequence with the adapter sequences at both ends was obtained.

TABLE 3
50 μL of Tn5 transposase (V50 Tn5 Enzyme 5)
enzyme digestion reaction system

| | Volume | Final |
|---|---|---|
| Components | (μL) | concentration |
| DNA fragment | | [symbol] 50 ng |
| TTE Mix V50 Tn5 Enzyme | 5 | |
| TTBL 5X Reaction Buffer | 10 | 1× |
| ddH₂O | | |
| Total volume | 50 | |

PCR amplification was further conducted by taking the fragmented DNA with the adapter sequences at both ends as a template and N5 (5′ AATGATACGGCGACCACCGAGATCTACACTAGATCGCTCGTCGGCAGCGTC3′) and N7(5′ CAAGCAGAAGACGGCATACGAGATTAAGGCGAGTCTCGTGGGCTCGG3′) as primers. The PCR system is shown in Table 4 and the PCR procedure is shown in Table 5. A PCR product stripe was verified by means of run-the-gel of a 1% agarose gel, and the gel was cut to recover the stripe with a fragment size distributed at 100-300 bp. The stripe was recovered with a PCR cleanup Kit, eluted with 50 μL of ddH₂O and quantified with Qubit.

TABLE 4

| | Volume | Final | |
|---|---|---|---|
| Components | (μL) | concentration | |
| Q5 High-Fidelity DNA | | 0.02 | U/μL |
| Polymerase | 0.5 | | |
| 5xQ5 Reaction Buffer | 10 | 1× | |
| DNA template | 9 | | |
| N5 primer | 2.5 | 0.5 | μM |
| N7 primer | 2.5 | 0.5 | μM |
| dNTP Mix | 1 | 0.2 | mM |

| | |
|---|---|
| ddH₂O | 24.5 |
| Total volume | 50 |

TABLE 5

| Temperature | Time | |
|---|---|---|
| 72° C. | 5 | min |
| 98° C. | 30 | s |
| 98° C. | 10 | s |
| 63° C. | 30 | s |
| 72° C. | 15 | s |
| Number of Cycles | 30 | |
| 72° C. | 5 | min |
| 4° C. | 10 | min |

c) The fragmented DNA sequence was obtained by using the adapter sequences to generate the fluorescent-labeled probes

PCR amplification was conducted by taking the fragmented DNA sequence with the adapter sequences at both ends as a template and TAMRA as fluorescently-labeled primers F5′ TAMRA-TCGTCGGCAGCGTC AGATGTGTATAAGAGACAG3 and R (5′ TAMRA-GTCTCGTGGGCTCGG AGATGTGTATAAGAGACAG3′). The PCR system is shown in Table 6 and the PCR procedure is shown in Table 7. The fragmented DNA sequence was recovered with a PCR cleanup Kit, eluted the same with 22 μL of ddH₂O, and quantified with Qubit.

TABLE 6

| | Volume | Final | |
|---|---|---|---|
| Components | (μL) | concentration | |
| Premix Taq Hot | 10 | 1× | |
| Start Version | | | |
| DNA template | | <250 | ng |
| TAMRA labeled F | 2.5 | 0.5 | μM |
| TAMRA labeled R | 2.5 | 0.5 | μM |

| | |
|---|---|
| ddH₂O | |
| Total volume | 50 |

TABLE 7

| Temperature | Time | |
|---|---|---|
| 94° C. | 10 | s |
| 55° C. | 5 | s |
| 68° C. | 20 | s |
| Number of Cycles | 35 | |
| 68° C. | 5 | min |
| 4° C. | 10 | min |

d) Alcohol Precipitation of the Probes

The product obtained in Step c) was merged and then subject to alcohol precipitation. An alcohol precipitation system is shown in Table 7. The solution was mixed uniformly and placed overnight at 80° C. below zero; the probes were recovered, centrifugalized at 13,000×g at 4° C. for 20 min, washed with 70% ethanol once, and centrifugalized at 13,000×g at 4° C. for 5 min. Ethanol was removed carefully, and the solution was kept in dark at room temperature for about 5 min till the edge of the precipitate is transparent. DNAs were resuspended with 20 μL of hybridization buffer, and stored in dark at −20° C. for later use. The purified probes solution (the solution is the hybridization buffer) for the target on nucleic acids of interest was named as the Tn5 probes.

2. Preparation of BAC Probes

BAC probes were prepared according to the BAC probes preparation method in the paper “Robust 3D DNA FISH Using Directly Labeled Probes” published in 2013.

3. A Method of Conducting Hybridization Assay by Using the Tn5 Probe in Step 1

a) Cells pre-treatment: The K562 cells were subjected to three passages (referring to passage step) after recovery and the healthy cells were selected, washed twice after being resuspended with PBS after centrifugalizing, and transferred to a 1.5 ml EP tube, wherein each tube has about 5×10⁷ K562 cells. PBS was removed, 1 ml of 4% paraformaldehyde was added, and the cells were treated at room temperature for 15 min after being resuspended and mixed uniformly. The cells were centrifugalized at room temperature, 300×g for 3 min, and washed with PBS twice after paraformaldehyde was removed to finish pre-treatment of the cells;

b) The K562 cells were immobilized with 4% paraformaldehyde at room temperature for 10 min, and washed with 0.1M Tris-HCl for 10 min; then a membrane was penetrated with 0.5% Triton-X 100 containing 10 μg/mL RNase A and RNAs were digested; the cells were subject to water bath treatment at 37° C. for 30 min, washed with a PBS for three times and treated with a 0.1M hydrochloric acid solution at room temperature for 30 min. After being washed with PBS for three times, the cells were treated in a 50% deionized formamide 2×SSC solution at room temperature for 30 min, and dried after gradient ethanol dehydration. 10 microliter of mixed solution was prepared from the Tn5 probes solution prepared in Step 1 and the BAC probes solution prepared in Step 2, wherein a solute of the mixed solution was the Tn5 probe (final concentration is 5 ng/μL) in Step 1 and the BAC probe (final concentration is 2 ng/μL) in Step 2, and a solvent was the hybridization buffer. The mixture was sealed in the glass slide with Fixogum after the mixed solution and the K562 cells were mixed, and placed in a hybridization instrument for hybridization (at 75° C. for 5 min, and at 37° C. for overnight). The K562 cells were washed for three times at room temperature with a 0.3% NP-40 2×SSC solution the next day, 5 min every time, and then mounted with the DAPI-containing AntiFade mounting agent; the solution was sealed at the edge of the glass slide with Fixogum, and kept in dark at 4° C. or imaged directly.

c) The glass slide was sealed circumferentially with a mounting gel, and stored or directly imaged at 4° C. after solidified. Fluorescence microscope imaging and processing: The sealed glass slide was photographed with the SIM microscope. It is a Structured Illumination Microscope (SIM) (model: SIM (Structured Illumination Microscope), Nikon Ti-E automatic inverted microscope with an Andor Technology EMCCD camera (iXON DU-897X-9255)) of Nikonused in the present invention, equipped with 405, 488, 561 and 647 laser and corresponding optical fiber combinations, with CFI Apochromat TIRF 100× oil immersion lens. The lens oil is immersion oil and the refractive index is 1.515. The picture collecting software is NIS-Elements AR 4.3, and the processing software is Imaris 2.0.

The result is shown in FIGS. 5a-5d. In FIGS. 5a-5d, from left to right, FIG. 5a shows labeling the genetic site (red) by the Tn5 probes in Step 2, FIG. 5b shows labeling the genetic site (green) by the BAC probes in Step 2, FIG. 5c shows labeling cell nuclei by using DAPI and FIG. 5d shows a merged graph of three channel images. It can be seen from FIGS. 5a-5d that the signal of the Tn5 probes is strong and sharp. When labeling the same gene site with the BAC probes, the signal is co-positioned, illustrating that Tn5 probes can be prepared by using a small quantity of DNA templates. The labeling effect is consistent with that of the BAC probes, and the Tn5-FISH method has labeling specificity.

### Embodiment 2

In the K562 cells, Tn5-FISH and BAC FISH were combined to verify the resolution of 1 KB.

The experimental result is shown in FIG. 3. FIG. 3 is a combination of Tn5-FISH and BAC FISH in the K562 cells to verify 1 Kb genomic resolution. A schematic diagram of BAC (green) capable of covering a target labeling position (chr12: 53,250,000-53,280,000) and a schematic diagram of a region (solid red) where the Tn5 probes is designed are located on the upper side of FIG. 3; a labeled image of the Tn5 probes with a total length of about 4 Kb is located on the left lower side, wherein a red signal point is a Tn5 probes signal, green is a BAC probes signal, and the big figure is a multichannel combined image where a blue region represents a cell nucleus dyed by DAPI; and a labeled image of the Tn5 probes with a total length of about 1.2 Kb is located on the right lower side, wherein a red signal point is a Tn5 probes signal, green is a BAC probes signal, the big figure is a multichannel combined image where a blue region represents a cell nucleus dyed by DAPI. The labeling effects of two regions with different lengths were verified by adopting Tn5-FISH and BAC FISH respectively. It was found that the signal of Tn5-FISH and the signal of BAC FISH were co-positioned well. Meanwhile, the DNA length of the Tn5-FISH probes template was about 1 kb, illustrating that the Tn5-FISH had a labeling capacity of 1 Kb resolution in a genome, superior to the genomic resolutions of various FISH methods reported before (Oligopaint resolution is 4 Kb, MB-FISH is 2.5 Kb, HD-FISH is 3.5 Kb and CasFISH is 10 Kb).

The specific experimental method is as follows:

1. Preparation of Tn5 Probes

a) Acquisition of the DNA Fragment of the Target on Nucleic Acids of Interest

A DNA sequence (as an initial sequence) containing a genetic site chr12: 53,250,000-53,280,000 was downloaded from the UCSC website (hg19). The undesired sequence was excluded from the initial sequence such as an AT-rich sequence (300 bp) and repeated sequence (1180 bp), and three primer pairs were designed in a position without the repeated sequence:

a primer pair 1: primer IF:
5‘ACCAGGCTTGGCCTACTAGA3‘ and primer
1R: 5‘CTTCCTGGAAGAATGGTCTTC3‘;
a primer pair 2: primer 2F:
5‘CTCAGGTCTATGCCTGCATC3‘ and primer
2R: 5‘CATATGGTTTCTGTATGGCTCC3‘;
a primer pair 3: primer 3F:
5‘TGAGCGCCTTAGCCAGGAGT3* and primer
3R: 5‘GAAGGCACAGGGTTGGAGGT3‘

The genomic DNAs of the K562 cells system as a template and the primer pair 1 as primers were subject to PCR to obtain an amplified target DNA sequence of interest 1, wherein the sequence of the target DNA sequence 1 is shown in sequence 2. The amplified PCR product was collected and was ready for next operation.

Similarly, the target DNA amplified from primer pair 2 and primer pair 3 can be obtained following the same procedure. The genomic DNA of the K562 cells system as a template and the primer pair 2 as primers were subject to PCR to obtain a target DNA sequence of interest 2, wherein the sequence of the target DNA sequence 2 is shown in sequence 3.

And the genomic DNA of the K562 cells system as a template and the primer pair 3 as primers were subject to PCR to obtain a target DNA sequence of interest 3, wherein the sequence of the target DNA sequence 3 is shown in sequence 4. Probes for the target DNA sequence 3, the target DNA sequence 3 and the target DNA sequence 3 respectively were obtained by continuous operation according to the method with Step b) and Step c) in Step 1 in Embodiment 1.

The three obtained probes were tested continuously according to the method in Step 3 in Embodiment 1. The result is shown in FIG. 3.

### Embodiment 3

Tn5-FISH and traditional BAC FISH were combined to verify labeling specificity of Tn5-FISH to the genomic site in WT mESC cells and Platr22-KO mESC cells.

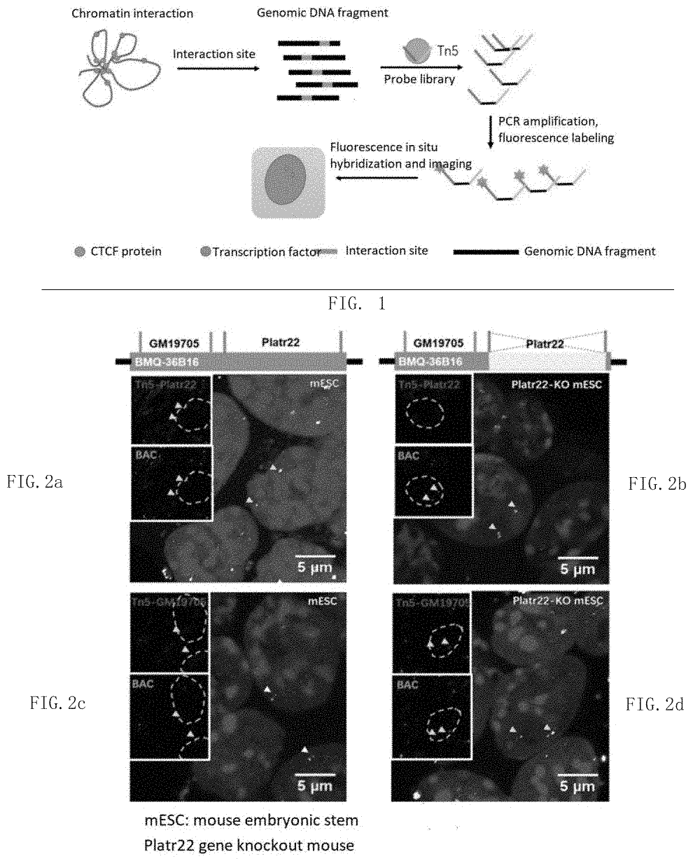

The experiment result is shown in FIGS. 2a-2d. FIGS. 2a-2d is a comparison of combination of Tn5-FISH and traditional BAC FISH to verify labeling specificity of a Tn5-FISH genomic site in WT mESC cells and Platr22-KO mESC cells, where the BAC probes (green) and the Tn5-Platr22 probes (Red, FIG. 2a, FIG. 2b) or the Tn5-GM19705 probes (red, FIG. 2c, FIG. 2d) were hybridized simultaneously in the WT mESC cells (FIG. 2a, FIG. 2c) or Platr22-KO mESC cells (FIG. 2b, FIG. 2d). That is, FIG. 2a shows simultaneous hybridization of the BAC probes (green) and the Tn5-Platr22 probes (red) in the WT mESC cells, FIG. 2b shows simultaneous hybridization of the BAC probes (green) and the Tn5-Platr22 probes (red) in the Platr22-KO mESC cells, FIG. 2c shows simultaneous hybridization of the BAC probes (green) and the Tn5-GM19705 probes (red) in the WT mESC cells, and FIG. 2d shows simultaneous hybridization of the BAC probes (green) and the Tn5-GM19705 probes (red) in the Platr22-KO mESC cells.

In the WT mESC, two Tn5-FISH signals of adjacent genomic sites GM19705 and Platr22 that are 6.9 Kb away from each other were co-positioned well with the BAC FISH signal covering the two regions. However, in the Platr22-KO mESC cells, only the GM19705 site was co-positioned well with the BAC FISH signal. The Tn5-FISH signal in the Platr22 site was not detected, and there was only the BAC FISH signal. The experiment illustrates that the Tn5-FISH label is of very good specificity.

### Embodiment 4

In the GM12878 cells, multicolor Tn5-FISH was used to verify the predicted interactions of sites at both ends of chr2: 227672028-227743852.

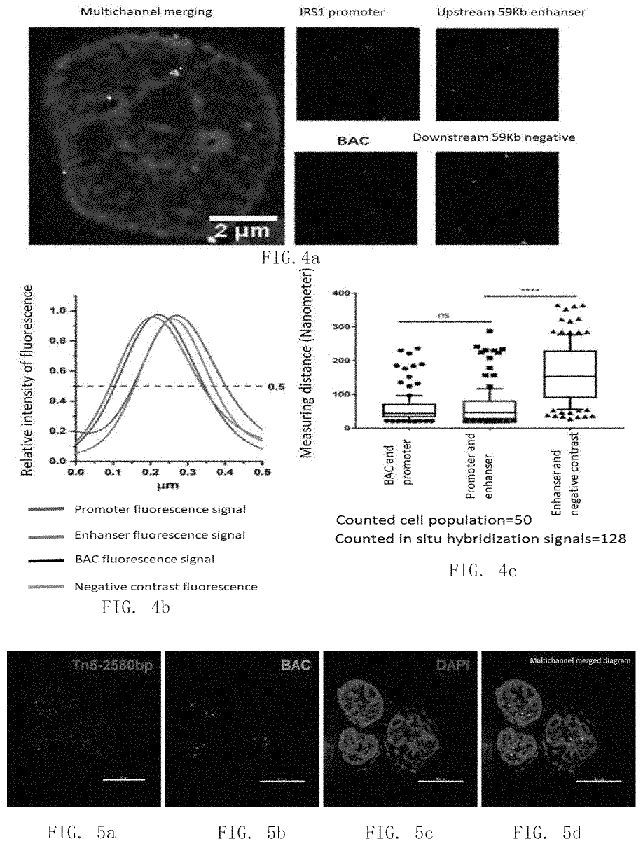

According to ChIP-seq data and Hi-C data, the two sites (59 Kb away from each other with E-P interaction) (promoter and enhancer) located at both ends of chr2: 227672028-227743852 in the GM12878 cells were verified, and the site (negative control) with a reverse same distance was used as a reference. Meanwhile, the BAC probes capable of covering three sites simultaneously were provided as reference. The result is shown in FIGS. 4a-4c. FIGS. 4a-4c is a verification of predicted interaction of interaction sites located at both ends of chr2: 227672028-227743852 in the GM12878 cells by multi-colored Tn5-FISH, where FIG. 4a (four-color hybridization image) shows that a promoter (chr2:227672028-227684087) (magenta) at 16 Kb upstream of a transcription initiation site of an IRS1 gene, an enhancer (chr2:227731793-227743852) at 59 kb upstream and a spot of the negative control (chr2:227612297-227624299) (green) with the interaction (yellow) with the promoter or without interaction with the promoter at 59 kb downstream are limited to the traditional BAC FISH (Alexa Fluor 594, red) spatially, and merge represents a co-positioned signal; FIG. 4b is a fluorescence intensity peak of an in situ hybridization signal point, and the spatial resolution of Tn5-FISH in FIG. 4b is about 250 nm; FIG. 4c is a spatial distance distribution diagram between the measured in situ hybridization signal points, and statistical analysis of the spatial distance between the Tn5-FISH spots in FIG. 4c shows that the predicted E-P distance is shorter than that of the negative control. It can be seen from FIGS. 4a-4c that Tn5-FISH probes in three colors were prepared at three sites with fragments with a length of 2 Kb respectively, and signal points in the cells were gathered together after the three probes were hybridized. By selecting a fluorescence labeling signal center point of each site and measuring the spatial distance therebetween, it was found that distance distribution from the promoter to the enhancer was smaller than the spatial distance from Site1 to Site3, and the two had a significant difference, indicating that Tn5 is quite suitable for analyzing interactions of chromatins that are separated at 100 Kb and within 100 Kb, and traditional BAC FISH is often not suitable for labeling and analyzing the distance.

Finally, it should be noted that the above embodiments are only used to explain the technical solution of the present invention, not constrained as limitation. Despite the present invention is detailed by reference to the aforementioned embodiments, it will be understood by those skilled in the art that they may still make some modifications to the technical solution recorded by the aforementioned embodiments or make equivalent substitutions on part or all of technical features therein. Such modifications or substitutions do not deviate the nature of the technical solution from the scope of the technical solution in the embodiments of the present invention.

### INDUSTRIAL APPLICATION

The present invention is a fast and efficient FISH method which requires a small quantity of templates and has high genomic resolution without a need of Cot-1 DNA, serving as an optional alternative solution for the current traditional FISH technical solution. Particularly, the method has a high resolution which is 1-2 orders of magnitude (as shown in FIGS. 2a-2d) higher than that of the traditional FISH technique. Therefore, it can be applied to many places where the detection failure or wrong detection of traditional FISH may occur as a result of insufficient resolution.

1. A method for preparing probes for targets on nucleic acids of interest, characterized in that it includes:
a) obtaining a target DNA sequence of interest;
b) using transposase to fragment the target DNA sequence and to add adapter sequences to both ends of the fragmented DNA sequence; and
c) using the adapter sequences to obtain the fragmented DNA sequence to generate the probes.

2. The method according to claim 1, characterized in that the target DNA sequence of interest is obtained by excluding the region containing the undesired sequence from the initial sequence.

3. The method according to claim 2, wherein the regions of the undesired sequences are selected from the repetitive sequences, conservative sequences, GC-rich sequences, or AT-rich sequences.

4. The method according to claim 2, wherein the method of exclusion is to amplify the target DNA sequence of interest.

5. The method according to claim 2, wherein the region of the undesired sequence is 100 bp at least.

6. The method according to claim 1, wherein the transposase is selected from one of or any combination of Tn1, Tn2, Tn3, Tn5, Tn6, Tn7, Tn9, Tn10, Tn551, Tn971, Tn916, Tn1545, Tn1681, Tgf2, Tol2, Himar1 and HARBI1.

7. The method according to claim 1, wherein the probes are labeled.

8. The method according to claim 7, wherein the label is selected from the group consisting of fluorophores, colorimetric labels, quantum dots, biotin, alkyne groups for Raman diffraction imaging, cycloolefins for click reaction, priming groups for polymer labeling, polypeptide/protein molecules, LNA/PNA, unnatural amino acids and their analogs, unnatural nucleic acids and their analogs, and nanostructures mentioned above.

9. The method according to claim 8, wherein the nanostructures include inorganic nanoparticles, NV-center, aggregation/assembly induced emission molecules, rare earth ion ligand molecules, and polymetallic oxygen dusters.

10. The method according to claim 1, wherein in step c), the method for generating the probes is to amplify the fragmented DNA sequence by using the primers capable of binding to the adaptor sequence.

11. The method according to claim 10, wherein the primer is labeled.

12. The method according to claim 11, wherein the label is selected from the group consisting of fluorophores, colorimetric labels, quantum dots, biotin, alkyne groups for Raman diffraction imaging, cycloolefins for click reaction, priming groups for polymer labeling, polypeptide/protein molecules, LNA/PNA, unnatural amino acids and their analogs, unnatural nucleic acids and their analogs, and nanostructures.

13. The method according to claim 12, wherein the nanostructures include inorganic nanoparticles, NV-center, aggregation/assembly induced emission molecules, rare earth ion ligand molecules, and polymetallic oxygen clusters.

14. A method for performing a hybridization assay, which includes generating probes and making the target nucleic acids contact the probes by using the method claim 1.

15. The method of hybridization assay according to claim 14, wherein the hybridization assay is in situ hybridization.

16. The method of hybridization assay according to claim 15, wherein the probes include one, two or more than two kinds.

17. The method of hybridization assay according to claim 15, wherein when there are two or more than two kinds of probes, each kind of the probes will display a different color after being labeled.

18. The method of hybridization assay according to claim 15, wherein the in situ hybridization is 3D FISH labeling the probes to the fixed target cells.


SEQUENCE LISTING (4 sequences)

SEQ ID NO: 1 - DNA, 2580 bp, Artificial Sequence
  Synthesized
atccttccagtgttaggttgatggtggctaggactcatcttctcaagaagattctatttctagagagcgtgggctgggtcttgccttgccagtgtccccagactgccatgcccggcacactgccctgcgtgctttgggtacaacccatgcttcggctgcagcagtggggagacttccatttggccttattgcgttccctgccatcaggggatgagaggctcccctggcagaggcgtcccattccttctaagtcccagtccttctaagtccttctaagttcccttgaaggacaaggccgctcaaagcaaaaaagctggctgtcctccactttggaggccttgaaaacctttatgaatgttttgctctggggaaggcaggctctggggagatctcagacctcagctaagaagatgctccccatccagggctggatggtcccagctcccctgactgtcgtggccagaccggccatgtcggacgcctcctgaccccagtgtgagcctgagtttaaaggaagtttgaagaggagagaaatgtgtgaagaggaaaaaacaagtttcttacatatgtttactggtccaacaccttcacagagcctggaaaggggaagaaacaaatcatttcacatgcactattgtttcctgaaaacctatttgccataaaaagggctttcttgtttgagtagatctgtggagcagtagttctcacccctggttatacacacatctggggagcgttgaaaaacacgttcttagaccctatcccaaaaccattagattaaaatatttaaggaaatttatcattagtagtaattttagcaaaattactaatgattctggtgcagcttatctagacttgcatttgagaatcacaggtctagagccttcttctgattccctgacaagggcaatgcaactaatgtttgcagagaaggtattgtcatctttaaagactatgtatttctgcagttcttcactgtccccttctcttccttagtctttctagatgcctaagtctgacttcactctcctgctggactgagagtactcaccggctctcctcgccctccagcaggcgcctgtaggtggcgatctcgatgtccaggcccagcttggcattcatcagctcctggtactcgcacagctgccgcgccatgtcctgcttggcctgctgcagggcacactccagatctgccagcttgcatttggcatcactgagggtcgcctcgccctgctgctcggcctcggccactgcagcctccaacttggcacgctgcagggaaggcagtttgcaggtaggcttcggcagtgccagggcatccccaggttgggttagaaacacggcccctcttagtgggaacatagcctggggccaaggcagggatggaccccatggtggctgctttcccaggcactggagaccctgtagtttgatgtcactatggggtagggcattgctgttgtttccatccccactgactttgtcactgctgatttcagctgccccagagtcatggcatctagagggtaaatttattctttctgtaccaccttccctgctgggtctgctctagccctttacttctatgtgggaagctctccttcccttccatatttttatcctccagaaagccttcctggactgatccctccattccttggtttctgggtcttgatggtctccctcacctcattagacttgggggttcccaaagcctgaggccctggttctccctcatctgaggctttccctcctttcctttgcccacctgagccttggcgtgctcaatctctgccttaagcctctggatcaggcgggtcagttcgttgatctcgttccgtatgttgcgcaggttgtcacagtgttggccagctgtcacctgcatctcttcatactgcggagagaggagggtattttgagggttcggaggtcaggtcaggatcacagcccccactgagttgggggcttcaaacctccctgggcctgatctgttatatatgagtcattcccagagctgcctacatcccttccccagcctccactgcccaccttggtctggtaccaggcctcagcatcagcccggctgcgcctggccacctcctcatactgggccttgacctcagcaatgatcccatcaaggttcaggtcacggctgttgtccatcttcacaatgaccgacgtctctgagatgtgcgactgcagcaactggatttcctgtgttggaggattggggagacagatgtgcttagagtctcatttagtagctgggactctgccacagggggcccaggaagtggtgccaggggcagagagcatcactcattggttcataaaaaagcaagaccatctaaattgatgataaagaaaaagacatgtctgagaaccatctcaggctgactttagagctcttgtgatctactcagaaacatccctttaacacactactttgctcagtaagtattaaaaccatggggatctaagaacatggtggaagaaagtgggagccactctagggcttaaacttttcttagattatctacaaagtggctgggctggtcacagaccgttgagacctgacaa

SEQ ID NO: 2 - DNA, 1170 bp, Artificial Sequence
  Synthesized
accaggcttggcctactagatgctgacttgcctggatctcctcttgctctcactgtcatctgaggacaaccatcccttctggctccctgagatgagaggggaagagacagagagaaagaaagaaaatgtgtagggacaagggtatacagggagcttgtaagatgtgtgtaaataaatggggggggagcatgagtgcgaaggtgcagagatcatctatgatcatgcaggggcatgtgcgggccagtaaaatcacaggggctatgaaagtgacacagttatgtgtgagtgcgccaggcatcggtgtatgtggaagggtggggtgggaatgtgtaccttctgggatgggtatagtggatgcttgcatgcatcagtgtgcaagggtatgtgtgagaaagacagaagctaggcatgtgtgtatgggggtgtgttataaatgtgcaggtgcgggtgtgtgaaggttgtgtgtgaatgttcaccttccccacctccaccccaaacacatatctcttggtaagggggcttcaacagagacaaaagtaccccaggctgagagaacagtgttcctgactctggaacaaacgatatttttctctttcaaagctttcagatttcctgaagaagagctgctagctcaaccaagctcagccttaaacccgcccctctgatcgaaactaaggaaggaaaccagcaccagccacccccgccctgcacccccacccctgccagggaatgctctatgccagcagaacgcacgccacgccccccatttctccctccccgcttgggcccacgtgctctccccagccactcagggtgcccagggcagctcccaaccaccaacagaagtttaagcccctctccaggctttgggagcactttgctacctgcccaggcaggcactaccctagggcaagcaggccactccctgccagggggaactgagaggtggggggtagggggtggcagcttcagaacacacaaccaacccccttcccgggaggcaggcactccaccccaccccgcatctcttcaagaagacaaaatcaggaaagagggctgggtggggagggcatctcaggaggaaagggtcacttagaggcagggtttgaatgtcaagaatgtcccttcctttcattcaagggtcccaaagaagaccattcttccaggaag

SEQ ID NO: 3 - DNA, 1441 bp, Artificial Sequence
  Synthesized
ctcaggtctatgcctgcatctgtctgttctccagctggaatagccacccctccaccagaggggatggaggaatccaaagagagaatgcactcaggccagatggggcaactagcagagccatgggctgggcagctggtgaaggctccagtgaaggggagtgtaaggggcctcgggaagaaagggagagagattgttaagtgaaagaagggatgaggaggggggccaggcctctgtgccacctgattgagagggcaggcgcatggttgcatgcagtgtgggagggaatagggtgcagagatggtggaaaggaggcctgtggctgagcccaagtgcctacttccctgtgctgtcactgccccagggaggcctgggccatttgcctcaaagccagtggtgggccaggggcctcagaccccaaggagtgtacatgctgggagcttcctgagcaaccaagagcctccaaactctggaacgtgccagggtttcattcctggagccacctcattcccagccacagagaggagtgctcaagccccaagggtctccagggaagagaaactcagtgggactccagagtgaccagccaagaaggttcccagagggggaacagagaagaggggcacctggctggagattcaggagacaggagatgggcagggagcagggaaggtgcggaaagcagcagagacagcaggggaggggaaatgaagaatgtgcgaggaggaaactggaatgaaagtgccagctggggctggagcacatcactttgaggaggagacagagcaaacctcacctaaggccaccacccccagccccatcccagcttccccaggccctgctaagtgccaggaagaagcagtgtcaaatgcaaggaggagcagggcgtcagttccccacagccccttccaggtctctggagctctattccggaaaggagaggggaaaagtgtcacttgaagcccccaggcctcatgaaggccttttgcaagagggaggcacagaactacatcaaaccccctttaaaagtttcttggcaagcctgacccaccacagggtaataggctggtgctgccacaaaggggactgaagttaggtgactacagccatctctgaaaggacaaagtcaatatacagaaggactaggtccagggaccctggtcaggcagggataggtggccccatggaagctgggaaatagtcaaaatgtcttctctagatccccctcccacatttgaccctctgccccagtctttttgtgccaaaccacagcccagagtccccattctctgggacctctcagaggaactctgctctctaggtggtgggggtggggcccccgttacattattttcagactcactaaacacctattaatttgtagggggcaggaagactgggaaacactaatccaaccccatgggagccatacagaaaccatatg

SEQ ID NO: 4 - DNA, 1530 bp, Artificial Sequence
  Synthesized
tgagcgccttagccaggagtggcctccacctggccccacagcaaacctgggggaaactccaccacctccacagggagaccagccttcaacttcatccaggctttcccataccaagagaggatcccccaggactgtgagagggactggggtgggaaggagaaagaagccaagagtcagtacatgtggggtttggagtaggcaacaggatatgccagatcccggggctggggaagaggaccctgcagctcgacccacgcacactttgtaggacacacacacacactcacatacacatgcacaatggctgctggacacagtgcacagatccagctgtgggggtcattgcaaagatgcccagacacacacacgtgcatgtttcatgtggtgcacacctgatatatagagctgttaccaacagacatgaaggaatcagtggagtcattacacacagattcacaatggtgtgcaggtcattactcacacacacacacacacacacacacacacacacacacacacagtcagaaccaccaaaaagaggatgatgagggagtgagaagaacgtttgaagctgttgtaggaacaaaccagctcaggggcctgaccaaacccactcaagccccgacccagaaaacctagacctcttcctctgctccccacaggcactcacaatggacagcccaccagtgcccctctgccctctgggcctaagacccggactactgatgtacacacagaggaacaggcatgcacacacacatgcctttgagaaaacacctatgtgaacacaaactcacaacagatgtgttccccaaacataatatgcaaaagccacataggcctttccaacaagcatgtacacacaacagtcactcgggcgcacaaccatcccacaaacatatatccatgcaagactctcacacaagcacatccagtgccagtcacacaaacacacaaccacccatgcactcacacagacccatggatccatgtacacaacgtaggaaggaggaatacaaatgcacacacaaatcaaatgcacacgcaaatcaaatgcacacatgagttaaatgcactcacgatggggatatgtgtgcacagaccatgagcatgacacaccttcctcccacagcacgcaccactgcccctcaaccttccatcttggcacaaacaaggcctgggcttgctccatcagctccatctcctccatgcctgagacaggccctgcccaacctgccccagcctgcttgagtggggccccgcctgctcccagcaccagctgccaacccagctcagcacagggcaagctgcccactgctcgggcacgtcgctgccccaccccctcctccttaggcctgccatctcagcatctgggcctcagcctaggccgcacctagcccatttctgcaggtgagtgggtaggcagggtgcttgatccccacagctggaggtgaggaaagggaccaggcagttaagaccccacgaggaaggggcagcctggctcagagcctctcacctccaaccctgtgc